(19) United States
(12) Patent Application Publication
(10) Pub. No.: US 2012/0202242 A1
(43) Pub. Date: Aug. 9, 2012

(54) System and Method for Non-Sterile Heterotrophic Algae Growth

(76) Inventor: David A. Hazlebeck, El Cajon, CA (US)

(21) Appl. No.: 13/024,103

(22) Filed: Feb. 9, 2011

(51) Int. Cl. C12P 1/00; C12N 1/12; C12M 1/00

(52) U.S. Cl. 435/41; 435/289.1; 435/257.1

(57) ABSTRACT

A system and method for non-sterile heterotrophic algae growth is provided. The system includes a first Continuous Stirred Tank Reactor (CSTR), kept under sterile conditions, for mixing a full load nutrient with algae cells to create a culture. After being transferred to a first Plug Flow Reactor (PFR), a nutrient is depleted from the culture to create an effluent. By depleting the nutrient, algae cells are able to grow more rapidly without having to compete with other microbes for available nutrients. Once the effluent is created, it is transferred to a second PFR where organic carbon is added. By adding organic carbon, the algae cells grow intracellular oil rapidly. Algae cells are then removed for processing into biofuel.

### FIELD OF THE INVENTION

The present invention pertains generally to methods for growing algae. More particularly, the present invention pertains to a method for heterotrophically growing algae in a non-sterile environment. The present invention is particularly, but not exclusively, useful as a method for growing algae using a two-stage bioreactor wherein a first stage enables rapid algae cell growth under sterile conditions, and wherein a second stage stimulates rapid oil production in the algae cells under non-sterile conditions.

### BACKGROUND OF THE INVENTION

As worldwide petroleum deposits decrease, there is rising concern over petroleum shortages and the costs that are associated with the production of petroleum products. As a result, alternatives to energy products that are currently processed from petroleum are being investigated and commercially deployed. In this effort, biofuel has been identified as a viable alternative to petroleum-based transportation fuels.

One method of biofuel production currently in commercial use is the extraction of intracellular oil found within algae cells. Currently, the various processes used to create biofuel in this manner are quite expensive relative to the process of extracting and refining petroleum. Specifically, the conditions necessary to facilitate a rapid heterotrophic growth rate for algae cells in large-scale operations have been found to be expensive to create. In further detail, one reason for the high costs associated with the heterotrophic growth and harvesting of algae cells results from the need to maintain sterile conditions throughout the algae growth process. Sterile conditions are essential to heterotrophic algae growth to ensure that other microbes, specifically bacteria and fungi, are not present in the system to compete with algae cells for nutrients in an algal culture. Capital costs are high because of the cost of specialized equipment required to maintain a sterile growth environment. And, operational costs are high because of the significant amount of energy, usually in the form of heat, required to maintain a sterile growth environment. With costs associated with maintaining a sterile system being so high, algae cells cannot be produced on a large enough scale to make biofuel production cost-effective.

A further drawback of maintaining sterile growth conditions is that the system must be periodically shut down to sterilize all of the system components. While the system is shut down for sterilization, no algae cells are produced. Additionally, the need to periodically shut down the system means that only batch processing, as opposed to a continuous-flow method of processing, is available as an option for operation of the system. As understood in the trade, batch processing is less efficient than continuous-flow processing as only a finite amount of material can be processed by the system at one time.

In light of the above, it is an object of the present invention to provide a system and method for heterotrophically growing algae that is suitable for use in the production of biofuel. Another object of the present invention is to provide a system and method for heterotrophic algae growth that occurs in two stages with the second stage being conducted under non-sterile conditions. Still another object of the present invention is to provide a system and method for heterotrophically growing algae that reduces capital and operational costs by allowing the system to operate continuously. Yet another object of the present invention is to provide a system and method for heterotrophically growing algae in a non-sterile environment that is simple to implement, easy to use, and comparatively cost effective.

### SUMMARY OF THE INVENTION

In accordance with the present invention, a system and method for implementing non-sterile heterotrophic algae growth is provided. For the present invention, a two-stage process is implemented. Importantly, the first stage is conducted under sterile conditions, while the second stage is conducted under non-sterile conditions. In the first stage, a full load nutrient is mixed with algae cells in a chamber of a Continuously Stirred Tank Reactor (CSTR) to form a culture and promote rapid growth rate of the algae cells. Then, the culture is transferred to a first Plug Flow Reactor (PFR) having a chamber where a selected nutrient is depleted from the culture in order to create an effluent. At this point, the first stage is complete, and the effluent is transferred to a chamber within a second PFR to begin the second stage. While in the chamber of the second PFR, organic carbon is added to the effluent to rapidly increase the lipid content of the algae cells in the effluent. As a final step, the effluent is sent to a processor to extract the oil from the algae cells.

Structurally, a first inlet pipe is connected to the CSTR to allow for the addition of algae cells to the chamber of the CSTR. Also, a second inlet pipe is provided for adding a full load nutrient to the chamber of the CSTR. In addition, the CSTR and the first PFR are in fluid communication to facilitate the transfer of the culture from the CSTR to the chamber of the first PFR after mixing. For the present invention, the first PFR and the second PFR are connected by a transfer pipe to advance the effluent through the system. Also, a conduit is connected between the second PFR and a source of organic carbon to allow for the addition of organic carbon to the effluent while it is in the chamber of the second PFR. Additionally, an outlet pipe is connected to the second PFR to remove the effluent to a processor that will extract the oil from the algae cells.

Operationally, each of the two stages will have a residence time, with the first stage having a first residence time and the second stage having a second residence time. During the first residence time, at least one key nutrient other than carbon (i.e. nitrogen or phosphorous) is depleted from the effluent. By depleting one of these nutrients, other microbial contaminants will be unable to reproduce or survive. When fewer microbial contaminants are present in the culture, algae growth is enhanced as the algae cells do not compete with other organisms for the nutrients added to the culture. For the purposes of the present invention, the first residence time is envisioned to be in a range of 4-10 hours. During the second residence time, the only nutrient source added to the effluent is organic carbon. By only adding organic carbon to the effluent, the carbon to nitrogen (C:N) ratio will increase to a predetermined level. This predetermined level is calculated to stimulate rapid lipid growth (in the form of oil) within the algae cells. The carbon to nitrogen (C:N) ratio should be greater than 10:1. Preferably, it will be greater than 14:1 and in a range between 14:1 and 25:1. As envisioned for the present invention, the second residence time is envisioned to be in a range of 12-120 hours to allow for maximum oil production within the algae cells. For a preferred embodiment, the first stage and the second stage comprise a bioreactor. Within the bioreactor, the first stage is significantly smaller than the second stage when comparing the overall volume of each stage. Energy and equipment costs are reduced because the size of the first stage is smaller than the types of sterile systems currently in commercial use. At the same time, for the second stage, minimal energy and inexpensive equipment can be used because less energy and less expensive equipment is needed to maintain the algae cells under non-sterile conditions. An additional benefit of this configuration is that the algal culture in the second stage can be maintained at a lower cell density than in a system that is sterile throughout. Further, the lower cell density means dehydration of the organic carbon will not be required before it is added to the effluent. As a consequence, energy is conserved by maintaining the carbon in a liquid state.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic diagram of the layout of the system for the present invention; and

FIG. 2 is a flowchart illustrating the steps required for the system of the present invention.

### DESCRIPTION OF THE PREFERRED EMBODIMENTS

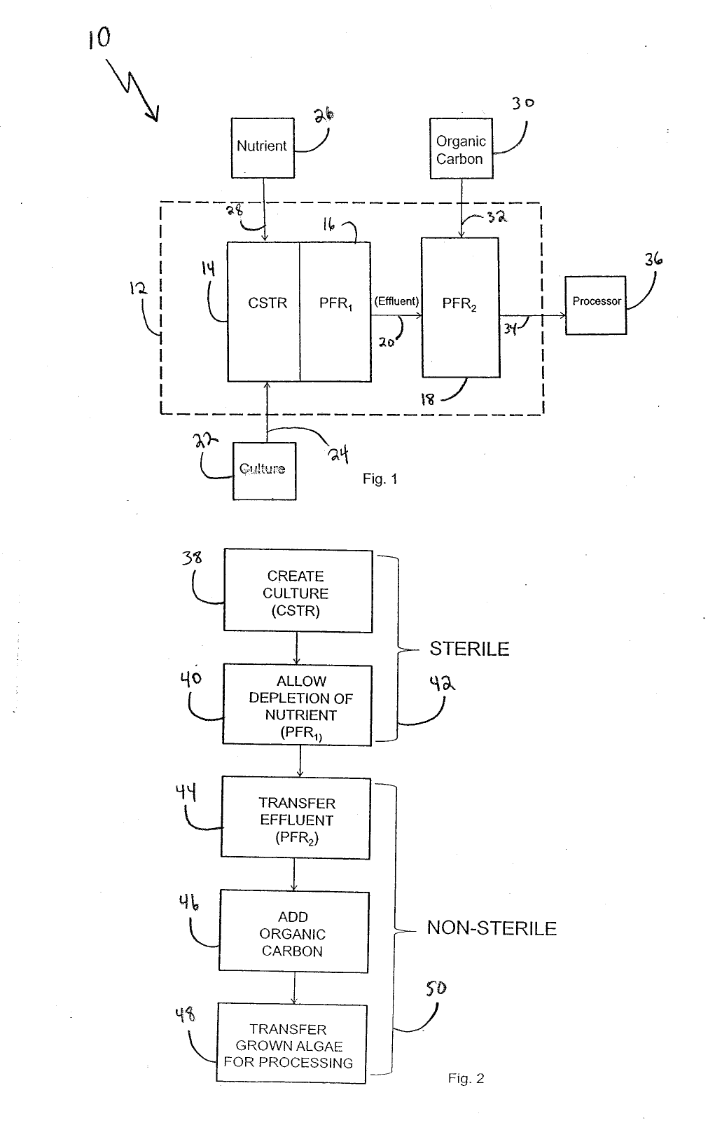

Referring initially to FIG. 1, a system as envisioned for the present invention is shown and generally designated 10. As shown, a bioreactor 12 is provided and has three major components: a CSTR 14, a first PFR 16, and a second PFR 18. Within the bioreactor 12, a transfer pipe 20 is also provided to connect the first PFR 16 and the second PFR 18.

Still referring to FIG. 1, additional components of the system 10 are located outside of the bioreactor 12 as shown. In particular, a culture source 22 is connected to the CSTR 14 by a first inlet pipe 24. In addition, a nutrient source 26 is also connected to the CSTR 14 by a second inlet pipe 28. An organic carbon source 30 is shown and is connected by a conduit 32 to the second PFR 18. Also, an outlet pipe 34 is connected between the second PFR 18 and a processor 36 that is used to separate the oil from algae cells as a first step in creating biofuel.

From an operational perspective, FIG. 1 shows that algae cells are moved from the culture source 22 to the CSTR 14 by way of the first inlet pipe 24. At the same time, a full load nutrient is transferred from the nutrient source 26 to the CSTR 14 by way of the second inlet pipe 28. For the system 10 of the present invention, continuous flow from the culture source 22 and the nutrient source 26 may be implemented. Further, the CSTR 14 is also operated continuously to facilitate the mixture of culture and nutrient. Once sufficient mixing has taken place, a culture is produced within the CSTR 14 and is transferred to the first PFR 16. While the culture is located in the first PFR 16, a selected nutrient (e.g. nitrogen or phosphorous) is depleted from the culture to allow algae cells to grow more rapidly. After the culture has been created in the CSTR 14 and grown in the first PFR 16, a first stage of the system of the present invention is complete. The amount of time the culture stays in the first stage is defined as a first residence time. Further, the first residence time is envisioned to be between 4 and 10 hours.

Again referring to FIG. 1 and the operation of the system 10 of the present invention, the now-depleted culture becomes an effluent and moves through the transfer pipe 20 to the second PFR 18. While in the second PFR 18 for a second residence time of between 12 and 120 hours, organic carbon is added to the effluent. Organic carbon flows from the organic carbon source 30 through the conduit 32. Once organic carbon is added, the carbon-to-nitrogen (C:N) ratio is increased to a predetermined level that will stimulate the algae cells in the effluent to rapidly produce lipids in the form of oil. The carbon to nitrogen (C:N) ratio should be greater than 10:1. Preferably, it will be greater than 14:1 and in a range between 14:1 and 25:1.

As a last step, the effluent is moved through the outlet pipe 34 to the processor 36. At the processor 36, an initial step in converting the algae cells into biofuel takes place. More specifically, the intracellular oil (lipid) is extracted from the algae cells for further processing.

Now referring to FIG. 2, the system 10 of the present invention is depicted as a two-stage process that comprises five steps in a preferred embodiment. As a first step, the culture is created in the CSTR 14 as shown in action block 38. Then, after transfer from the CSTR 14 to the first PFR 16, a nutrient (nitrogen or phosphorous) is depleted from the culture as depicted in action block 40. As shown, action blocks 38 and 40 represent the sterile stage 42 of the method of the present invention. Once the selected nutrient is depleted from the culture, an effluent is formed and is transferred to the second PFR 18 as illustrated in action block 44. While in the second PFR 18, organic carbon is added to the effluent as can be seen in action block 46. As a final step shown in action block 48, the oil-rich algae is transferred to the processor 36 to be harvested for use in creating biofuel. The steps in action blocks 44, 46, and 48 represent the non-sterile stage 50 of the method of the present invention.

While the System and Method for Non-Sterile Heterotrophic Algae Growth as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for implementing non-sterile heterotrophic algae growth which comprises:
a Continuous Stirred Tank Reactor (CSTR) having a chamber for mixing a full load nutrient with algae cells under sterile conditions to create a culture;
a first Plug Flow Reactor (PFR) having a chamber for receiving the culture from the CSTR and for growing the culture therein under sterile conditions during a first residence time, wherein a selected nutrient is depleted from the culture during the first residence time to create an effluent;
a second Plug Flow Reactor (PFR) having a chamber for receiving the effluent from the first PFR, and for holding the effluent therein during a second residence time;
a means for adding organic carbon to the effluent in the chamber of the second PFR during the second residence time to stimulate rapid formation of lipids within the algae cells; and
a means for removing grown algae from the second PFR.

2. A system as recited in claim 1 further comprising a processor for converting grown algae from the second PFR into a biofuel.

3. A system as recited in claim 1 wherein the means for adding organic carbon comprises:
a source of the organic carbon; and
a conduit connecting the source of organic carbon in communication with the chamber of the second PFR for adding organic carbon to the effluent.

4. A system as recited in claim 1 wherein the first residence time is in a range between 4 and 10 hours.

5. A system as recited in claim 1 wherein the second residence time is in a range between 12 and 120 hours.

6. A system as recited in claim 1 wherein depleting the selected nutrient inhibits growth of contaminants that compete with algae cells for food, and wherein the selected nutrient for depletion from the culture is selected from a group comprising nitrogen and phosphorous.

7. A system as recited in claim 1 wherein the second PFR is non-sterile.

8. A system for implementing non-sterile heterotrophic algae growth which comprises:
a sterile first stage having a Continuous Stirred Tank Reactor (CSTR) and a first Plug Flow Reactor (PFR), wherein a culture is created in the CSTR by mixing a full load nutrient with algae cells, and wherein the culture is transferred from the CSTR to the first PFR, and where algae cells in the culture are grown in the first PFR as a selected nutrient is depleted from the culture to produce an effluent; and
a non-sterile second stage having a second PFR, wherein the effluent is received from the first PFR, and further wherein an organic carbon is added to the effluent in the second PFR to stimulate rapid formation of lipids within the algae cells.

9. A system as recited in claim 8 further comprising a means for adding the organic carbon to the second PFR.

10. A system as recited in claim 8 wherein the organic carbon is added to maintain a predetermined carbon to nitrogen (C:N) ratio in a range between 14:1 and 25:1.

11. A system as recited in claim 8 further comprising a means for removing the grown algae from the second PFR.

12. A system as recited in claim 8 further comprising a processor for converting grown algae into a biofuel.

13. A system as recited in claim 8 wherein the first stage has a first residence time and the second stage has a second residence time.

14. A system as recited in claim 13 wherein the first residence time is in a range between 4 and 10 hours and the second residence time is in a range between 12 and 120 hours.

15. A system as recited in claim 8 wherein depleting the selected nutrient inhibits growth of contaminants that compete with algae cells for food, and wherein the selected nutrient for depletion is selected from a group comprising nitrogen and phosphorous

16. A system as recited in claim 8 wherein the first stage and second stage comprise a bioreactor.

17. A method for non-sterile heterotrophic algae growth which comprises the steps of:
mixing a full load nutrient with algae cells in a Continuous Stirred Tank Reactor (CSTR) under sterile conditions to create a culture;
receiving the culture in a first Plug Flow Reactor (PFR) wherein the first PFR has a chamber for receiving the culture from the CSTR and growing the culture therein under sterile conditions during a first residence time;
creating an effluent in the first PFR by depleting a selected nutrient from the culture during the first residence time;
transferring the effluent from the first PFR to a second PFR, wherein the second PFR holds the effluent for a second residence time; and
adding organic carbon to the effluent in the second PFR during the second residence time to stimulate rapid formation of lipids within the algae cells.

18. A method as recited in claim 17 further comprising the steps of:
removing the grown algae cells from the second PFR; and
converting the grown algae cells into a biofuel.

19. A method as recited in claim 18 wherein the adding step is implemented under non-sterile conditions.

20. A method as recited in claim 19 wherein depleting the selected nutrient inhibits growth of contaminants that compete with algae cells for food, and wherein the selected nutrient for depletion from the culture is selected from a group comprising nitrogen and phosphorous.
